(19) United States
(12) Patent Application Publication
(10) Pub. No.: US 2026/0240450 A1
(43) Pub. Date: Aug. 20, 2026

(54) CARE MONITORING DEVICE AND SYSTEM

(71) Applicant: DONGGUAN LELUX ELECTRONICS CO., LTD, Dongguan City (CN)

(72) Inventor: Wanfei LI, Dongguan (CN)

(21) Appl. No.: 19/181,069

(22) Filed: Apr. 16, 2025

(30) Foreign Application Priority Data
Feb. 14, 2025 (CN) 202510168118.3

(51) Int. Cl. A61B 5/103; A61B 5/00; G01L 1/22

(52) U.S. Cl. CPC A61B 5/1036; A61B 5/6891; A61B 5/6892; A61B 5/746; G01L 1/225; A61B 2562/0247

(57) ABSTRACT

The present disclosure relates to a care monitoring device and system. The care monitoring device includes an upper conductive assembly, a lower conductive assembly, an insulating assembly arranged between the upper conductive assembly and the lower conductive assembly, and a controller. The insulating assembly is provided with a hollow area. When a human body applies pressure to the upper conductive assembly, the upper conductive assembly and the lower conductive assembly are electrically connected through the hollow area in the insulating assembly, and the controller is configured to convert an electrical signal generated by the electrical connection into a pressure early warning signal.

### CROSS-REFERENCE TO RELATED APPLICATIONS

The present disclosure claims priority to Chinese patent application No. 2025101681183, filed on February 14, 2025, the entire content of which is incorporated herein by reference.

### TECHNICAL FIELD

The present disclosure relates to the field of fall prevention and rehabilitation equipment, and in particular, to a care monitoring device and system.

### BACKGROUND

Elderly people, severely bedridden patients, and patients with dementia are at high risk of accidental falls when lying in bed or sitting alone. The falls are often not discovered in time, especially at night or when they are alone, causing great harm. The above-mentioned groups have immobility or lack of normal self-care awareness, so they must be cared for by professional caregivers or family members for a long time, which brings great inconvenience and financial burden to their families. Therefore, a monitoring device is needed to monitor the user's activities.

### SUMMARY

One aspect of the present disclosure provides a care monitoring device, including an upper conductive assembly, a lower conductive assembly, an insulating assembly arranged between the upper conductive assembly and the lower conductive assembly, and a controller. The insulating assembly is provided with a hollow area. When a human body applies pressure to the upper conductive assembly, the upper conductive assembly and the lower conductive assembly are electrically connected through the hollow area in the insulating assembly, and the controller is configured to convert an electrical signal generated by the electrical connection into a pressure early warning signal.

In some embodiments, when a pressure applied by the human body to the upper conductive assembly disappears, the upper conductive assembly and the lower conductive assembly are electrically disconnected, and the controller is configured to generate a target early warning signal after the electrical connection is disconnected.

In some embodiments, the upper conductive assembly includes an upper conductive member and an upper plastic member, and the upper plastic member is located above the upper conductive member. The lower conductive assembly includes a lower conductive member and a lower plastic member, and the lower plastic member is located below the lower conductive member. The upper plastic member and the lower plastic member are configured to package the upper conductive member, the lower conductive member, and the insulating assembly.

In some embodiments, the upper conductive assembly includes an upper conductive member, the lower conductive assembly includes a lower conductive member, the care monitoring device is divided into two side regions and a middle region, the two side regions are configured to generate the pressure early warning signal, and the middle region is configured to generate the target early warning signal.

In some embodiments, in the two side regions, an upper circuit of the upper conductive member and a lower circuit of the lower conductive member are arranged in a staggered manner to form a bridge-type series connection.

In some embodiments, the upper circuit of the upper conductive member and the lower circuit of the lower conductive member overlap partially in a packaging direction of the packaged upper conductive member, the insulating assembly and the lower conductive member.

In some embodiments, in the middle region, an upper circuit of the upper conductive member and a lower circuit of the lower conductive member are arranged in an aligned manner to form a contact-type series connection.

In some embodiments, the side regions are configured to match positions of hands or arms of a user lying in bed or sitting on a chair, and the middle region is configured to match positions of shoulders or hips of the user, when the care monitoring device is used on a bed or a chair.

In some embodiments, the controller is configured to obtain a target duration in which the upper conductive assembly and the lower conductive assembly are electrically disconnect, and when the target duration is greater than a preset duration threshold, convert an electrical signal generated by the disconnection between the upper conductive assembly and the lower conductive assembly within the target duration into the target early warning signal.

In some embodiments, at least one of the upper conductive assembly and the lower conductive assembly is connected to the controller.

In some embodiments, the insulating assembly includes an insulating foam, and the hollow area includes gaps arranged in the insulating foam.

Another aspect of the present disclosure provides a care monitoring system according to any of the above-described embodiments and an alarm device.

In some embodiments, the alarm device includes a plurality of region indicator lights, and each of the region indicator lights corresponds to one care monitoring device. The alarm device is further configured to convert the target early warning signal received from the care monitoring device into a prompt signal according to a preset alarm prompt mode, and the prompt signal is configured to issue an alarm prompt according to the preset alarm prompt mode.

The details of the various embodiments of the present disclosure will be illustrated with the accompanying drawings and description below, based on which, other features, problems to be solved, and beneficial effects of the disclosure will be readily understood by those skilled in the art.

### BRIEF DESCRIPTION OF THE DRAWINGS

In order to describe the technical solutions of the embodiments of the present disclosure or the related art more clearly, the accompanying drawings required for describing the embodiments of the present disclosure or for describing the related art will be briefly introduced as follows. Apparently, the accompanying drawings, in the following description, illustrate merely some embodiments of the present disclosure, for a person of ordinary skill in the art, other drawings can also be obtained according to these accompanying drawings without making any creative efforts.

FIG. 1 is a schematic diagram showing a structure of an upper conductive assembly and a lower conductive assembly according to some embodiments of the present disclosure.

FIG. 2 is a schematic diagram showing a structure of an upper conductive member and a lower conductive member according to some embodiments of the present disclosure.

FIG. 3 is a schematic diagram showing an application scenario of a care monitoring system according to some embodiments of the present disclosure.

FIG. 4 is a schematic diagram showing a structure of an alarm device according to some embodiments of the present disclosure.

### DETAILED DESCRIPTION OF THE EMBODIMENTS

In the present disclosure, the terms “upper”, “lower”, “left”, “right”, “front”, “back”, “top”, “bottom”, “inner”, “outer”, “middle”, “vertical”, “horizontal”, “transverse”, “longitudinal” etc. indicate the orientations or position relationships as shown in the accompanying drawings and are merely used to illustrate the relative positional relationships between the components, and do not particularly limit the specific installation orientations of the components.

Moreover, some of the above terms may be used to express other meanings besides indicating the orientations or position relationships. For example, the term “upper” may also be used to indicate a certain dependency or connection relationship in some circumstances. For a person of ordinary skill in the art, the specific meanings of these terms in the present disclosure can be understood according to specific circumstances.

In addition, the terms “mount”, “arrange”, “provided with”, “connect”, and “couple” should be understood in a broad sense. For example, it may be a fixed connection, a detachable connection, or an integral structure. It may be a mechanical connection or an electrical connection. It may be a direct connection or an indirect connection through an intermediate medium. It may be an internal connection between two devices, elements or components. For a person of ordinary skill in the art, the specific meanings of the above terms in the present disclosure can be understood according to specific circumstances.

In addition, the structures, proportions, sizes, etc. drawn in the drawings in the present disclosure are merely used to match the contents disclosed in the specification so as to facilitate the understanding and reading of those skilled in the art, and are not used to limit the conditions under which the present disclosure can be implemented. Therefore, they have no substantive technical significance. Any structural modification, variation in proportional relationship or adjustment of size should still fall within the scope of the technical contents disclosed in the present disclosure without affecting the effects and purposes that can be achieved by the present disclosure.

The technical solutions of the embodiments of the present disclosure will be described clearly and completely below with reference to the accompanying drawings of the embodiments of the present disclosure. Apparently, the described embodiments are only part of but not all of the embodiments of the present disclosure. Based on the embodiments in the present disclosure, all other embodiments obtained without any creative efforts by a person of ordinary skill in the art fall within the scope of protection of the present disclosure.

The present disclosure provides a care monitoring device. The care monitoring device includes an upper conductive assembly, a lower conductive assembly, and an insulating assembly arranged between the upper conductive assembly and the lower conductive assembly. The insulating assembly is provided with hollow areas. An end of the upper conductive assembly and/or an end of the lower conductive assembly is connected to a controller. When a human body applies pressure to the upper conductive assembly, the upper conductive assembly and the lower conductive assembly are electrically connected through the hollow areas in the insulating assembly, and the controller is configured to convert an electrical signal generated by the electrical connection into a pressure early warning signal. It can be understood that the number of the hollow area can be one or more, depending on specific designs.

The upper conductive assembly refers to a part of the internal structure of a pressure sensing pad. The upper conductive assembly includes an upper conductive member and an upper plastic member. The upper conductive assembly is configured to form an electrical connection with the lower conductive assembly when pressure is applied, thereby achieving a closed circuit, and to disconnect from the lower conductive assembly when the pressure is removed or changed.

The lower conductive assembly also refers to a part of the internal structure of the pressure sensing pad. The lower conductive assembly includes a lower conductive member and a lower plastic member. The lower conductive assembly acts similarly to the upper conductive assembly. The lower conductive assembly remains separated from the upper conductive assembly in the absence of pressure, and contacts the upper conductive assembly to achieve a closed circuit when pressure is applied.

The insulating assembly refers to a layer of material located between the upper assembly and the lower conductive assembly. The insulating assembly is to configured to prevent the upper conductive assembly and the lower conductive assembly from directly contacting each other without external pressure. The insulating assembly is typically a layer of insulating foam with regularly arranged hollow gaps therein.

The hollow area refers to a preset opening or gap in the insulating assembly. The hollow area allows the upper conductive assembly and the lower conductive assembly to contact each other through the hollow areas when the pressure is applied, thereby achieving an electrical connection.

The controller is an electronic control unit that monitors and processes the signals generated by the lower conductive assembly and the upper conductive assembly. The controller can identify when the electrical connection occurs and generate a corresponding early warning signal or alarm signal accordingly. Optionally, the controller can further perform additional functions, such as delay processing to reduce the occurrence of false alarms.

The electrical signal refers to a current generated by a microcircuit loop formed when the upper conductive assembly and the lower conductive assembly come into contact through the hollow areas under pressure. The pressure change is converted into an electrical signal, which is received and processed by the controller.

The pressure early warning signal refers to a signal generated by the controller when a valid electrical connection event (i.e., a real pressure change rather than a false triggering) is detected. This signal is then sent to a receiving host alarm, which activates an appropriate alarm mechanism based on the signal received, such as a sound, a flashing light or a vibration prompt.

Specifically, first, the upper conductive assembly and the lower conductive assembly are formed, and a layer of insulating foam with regular hollow areas is sandwiched between the two layers, which together constitute the core component of the care monitoring device, i.e., a pressure sensing pad. Secondly, the controller is connected to the pressure sensing pad, and is responsible for receiving the electrical signal from the pressure sensing pad, and processing the electrical signal and converting it into the pressure early warning signal. Optionally, in order to improve the accuracy and reliability of the care monitoring device, the controller has a built-in algorithm to identify and filter out false trigger signals that occur under non-dangerous situations, such as false alarms caused by slight movements such as turning over or arm supporting.

In an example, when a user lies or sits on a bed, chair, etc., equipped with the care monitoring device, the body weight will be applied to the pressure sensing pad. If the user moves towards the edge of the bed or prepares to get up, a part of the body weight will be transferred to the pressure sensing pad, causing the upper conductive assembly to contact the lower conductive assembly through the hollow areas to form a microcircuit loop, thereby triggering an early warning signal (i.e., the pressure early warning signal). If the user completely leaves the pressure sensing pad, the original microcircuit will be disconnected, and the controller will detect this variation and output a more urgent warning signal indicative of human body’s leaving.

The care monitoring device not only directly solves the safety issues of the elderly and special groups at night or when they are alone, but also indirectly improves their quality of life, while providing caregivers and family members with more convenient and effective care methods. Through precise pressure sensing technology and intelligent signal processing, the care monitoring device provides efficient and reliable safety protection for people who need special attention. Furthermore, for some guardians who still have a certain degree of self-care ability, the care monitoring device can provide necessary protection without affecting their daily activities and help them maintain a certain degree of independence.

Optionally, when the pressure applied by the human body to the upper conductive assembly disappears, the upper conductive assembly and the lower conductive assembly are electrically disconnected, and the controller is configured to generate a target early warning signal after the electrical connection is disconnected.

The target early warning signal is a signal that is different from the pressure early warning signal and is more urgent than the pressure early warning signal. The pressure early warning signal is triggered when the user starts to move towards the edge of the bed or prepares to get up, but has not yet completely left the pressure sensing pad. The target early warning signal is activated when the user has left the pressure sensing pad, indicating that they may be trying to get up, fall, or leave a safe area.

Specifically, when a user lies or sits on a bed, chair, etc., equipped with the care monitoring device, their body weight is applied to the pressure sensing pad. In this case, the upper conductive assembly and the lower conductive assembly inside the pressure sensing pad are in contact with each other through the hollow areas in the intermediate insulating foam to form the microcircuit loop.

If the user completely leaves the pressure sensing pad (for example, trying to get up, accidentally falling, or leaving the safe area), the pressure originally applied to the pressure sensing pad will disappear. Due to the loss of the external pressure, the upper conductive assembly and the lower conductive assembly will be separated under the natural tension of the foam, causing the original microcircuit loop to be cut off, i.e., the electrical connection is disconnected. The disconnection of the electrical connection will be detected by the controller, which will then output the more urgent target early warning signal.

The mechanism as described above can thus respond quickly to the situation where the user leaves the safe area. Once the care monitoring device detects that the user has moved away from the pressure sensing pad and the pressure disappears, the target early warning signal is immediately generated, ensuring that the caregivers or family members can be notified as quickly as possible and take necessary actions. Especially at night or when the user is alone, the care monitoring device greatly improves the level of safety protection for the user.

In an embodiment, the upper conductive assembly includes an upper conductive member and an upper plastic member. The upper plastic member is located above the upper conductive member. The lower conductive assembly includes a lower conductive member and a lower plastic member. The lower plastic member is located below the lower conductive member. The upper plastic member and the lower plastic member are configured to package the upper conductive member, the lower conductive member and the insulating assembly.

The upper conductive member refers to a layer of conductive material located below the upper plastic member, which can generally be made of a specially designed conductive carbon circuit printed on a polyethylene-based film or a conductive aluminum foil. The upper conductive member is configured to contact the lower conductive member through the hollow areas in the intermediate insulating foam when the pressure is applied, to form the microcircuit loop.

The upper plastic member refers to a layer of synthetic polymer plastic covering the upper conductive member. The upper plastic member is configured to protect the internal conductive members from the external environment and facilitate the packaging of the entire pressure sensing pad to ensure its durability and reliability.

The lower conductive member refers to a layer of conductive material located above the lower plastic member, which is generally made of a polyethylene-based film with a specially designed conductive carbon circuit printed thereon or made of a conductive aluminum foil. The lower conductive member cooperates with the upper conductive member. When the lower conductive member contacts with the upper conductive member due to the external pressure, a complete circuit path is formed.

The lower plastic member refers to the synthetic polymer plastic at the bottom layer, which covers the lower conductive member. The lower plastic member not only serves a protective function, but also contributes to the overall packaging of the pressure pad, ensuring the integrity and stability of the sensor.

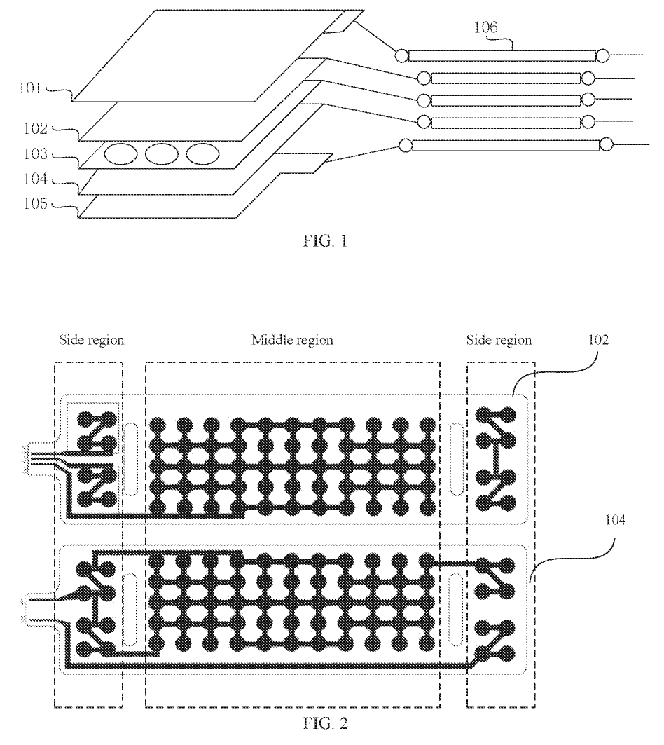

Specifically, referring to FIG. 1, the pressure sensing pad in the present disclosure uses a multi-layer composite structure to achieve efficient pressure monitoring. Specifically, the pressure sensing pad includes the two conductive assemblies and the insulating assembly, i.e., the upper conductive assembly, the lower conductive assembly and the insulating assembly.

The upper conductive assembly includes the upper conductive member 102 (e.g., a polyethylene-based film with a specially designed conductive carbon circuit printed thereon or a conductive aluminum foil), and the upper plastic member 101 (e.g., made of synthetic polymer plastic such as PVC or TPU) covering the upper conductive member 102. The upper conductive member forms a part of the circuit when pressure is applied, and the upper plastic member provides physical protection and contributes to the overall packaging of the pressure sensing pad.

The lower conductive assembly includes the lower conductive member 104 (made of the same material as the upper conductive member), and the lower plastic member 105 (also made of synthetic polymer plastic) located below the lower conductive member. The lower conductive member operates cooperates with the upper conductive member to achieve a closed circuit under an appropriate condition. The lower plastic member not only serves a protective function, but also assists in the overall packaging.

The insulating assembly 103 is a layer of insulating foam placed between the upper conductive assembly and the lower conductive assembly, with hollow areas arranged thereon, such as regularly arranged gaps. The insulating assembly 103 separates the upper conductive assembly and the lower conductive assembly from each other when no external force is applied to prevent false electrical signals, and allow the upper conductive member and the lower conductive member to contact under the pressure, thus forming the microcircuit loop.

The upper plastic member and the lower plastic member play a key role collectively, which jointly package the upper conductive member, the lower conductive member and the intermediate insulating assembly. The packaging method not only protects the internal components from external environmental influences such as moisture, dust, etc., but also improves the durability and reliability of the entire pressure sensing pad.

Due to the packaging of the upper and lower plastic members, the key components inside the entire pressure sensing pad (such as conductive members and insulating foam) are firmly fixed together. The packaging not only enhances the integrity of the structure, but also effectively prevents internal components from being displaced or damaged due to external factors (such as pulling or squeezing). Furthermore, the packaging ensures precise alignment between the conductive member and the insulating foam, so that when the external pressure is applied, the upper conductive member and the lower conductive member can accurately contact each other through the hollow areas on the foam to form the microcircuit loop. On the contrary, in a stress-free state, the two conductive members remain separated, thereby preventing false electrical signals. This design ensures the accuracy of signal transmission and reduces the possibility of false alarms.

In an embodiment, the packaged upper conductive member, the insulating assembly and the lower conductive member are divided into two side regions and a middle region. The two side regions are configured to generate the pressure early warning signal, and the middle region is configured to generate the target early warning signal. In some embodiments, the side regions are configured to match positions of hands or arms of a user lying in bed or sitting on a chair, and the middle region is configured to match positions of shoulders or hips of the user, when the care monitoring device is used on a bed or a chair.

The two side regions refer to parts located on the left and right sides of the packaged pressure sensing pad. It should be understood that the two side regions are specifically designed to detect, for example, if the user moves towards the edge of the bed or seat.

The middle region refers to a main central part of the pressure sensing pad, which is generally placed under the core of the user's body, such as the shoulders or hips. middle region is also a critical monitoring point in the entire system.

Specifically, after packaging is completed, the upper conductive member, the insulating assembly and the lower conductive member inside the pressure sensing pad are clearly divided into three functional areas, i.e., early warning regions on both sides and a middle leaving alarm region. Understandably, the regional design aims to provide accurate monitoring of different types of mobility behaviors and trigger the corresponding early warning signal.

The two side regions are located on the left and right sides of the pressure sensing pad, close to the edge of the bed or seat. The two regions are configured to mainly detect if the user moves towards the edge of the bed or seat, for example, if an arm or leg touches the edge. When the pressure is sensed in these regions, it means the user may be trying to get up or leave a safe position. The pressure early warning signals are generated from the two side regions. When the user's body (such as arms and legs) contacts any of the two side regions and applies a certain amount of pressure, even a slight change in pressure will trigger an early warning signal. This signal alerts the caregivers or family members that the user may be trying to get up or leave the safe position so that they can take actions to prevent potential risks.

The middle region is located in the middle of the pressure pad and is generally placed under the core of the user's body (such as the shoulders or hips). This region is a key point of the monitoring, which determines whether the user has completely left the safe position. Once the user moves away from the middle region, causing the applied pressure to disappear, it will be identified as a potential risk event. The target early warning signal is thus generated by the middle region. When the pressure in the middle region disappears (i.e., the user has left the middle region), the electrical connection between the upper conductive member and the lower conductive member is disconnected. In some embodiments, the controller will identify this change and delay the processing time by 3 to 5 seconds to determine that a leaving event has actually occurred rather than a brief action (such as turning over or arm supporting). If it is determined to be a real leaving event, a more urgent target early warning signal will be generated, indicating that the user may have stood up, fallen or left the safe area and require immediate attention.

Since the pressure sensing pad is divided into the two side regions and the middle region, the care monitoring device is able to distinguish different user behaviors. The two side regions are mainly configured to detect the behavior of moving towards the edge, which triggers an early warning signal, i.e., the pressure early warning signal. The middle region focuses on monitoring more serious completely-leaving event and generating a more urgent target early warning signal. This regional design improves the pertinence and accuracy of the early warning.

In an embodiment, in the two side regions, an upper circuit of the upper conductive member and a lower circuit of the lower conductive member are arranged in a staggered manner to form a bridge-type series connection.

The upper circuit refers to a conductive path located inside the pressure sensing pad and on the upper conductive member. The upper circuit is made of a specially designed conductive carbon circuit or a conductive aluminum foil, and cooperates with the lower conductive member. Under a specific condition (such as applying pressure), the upper circuit contacts the lower conductive member through the hollow areas on the intermediate insulating foam to form the microcircuit loop.

The lower circuit refers to a conductive path located inside the pressure sensing pad and on the lower conductive member. The lower circuit is made of a specially designed conductive carbon circuit or a conductive aluminum foil, and cooperates with the upper conductive member. Under a specific condition (such as applying pressure), the lower circuit contacts the upper conductive member through the hollow areas on the intermediate insulating foam to form the microcircuit loop.

The bridge-type series connection refers to a special connection method for the circuit, and refers to the method in which the upper circuit and the lower circuit in the two side regions are arranged in a staggered manner in the present disclosure. Specifically, the circuit on the upper conductive member is not directly aligned with the circuit on the lower conductive member, but the two circuits are arranged in a staggered manner, so that the two circuits can only come into contact with each other through the hollowed gaps in the intermediate foam and form an effective electrical connection when they are subjected to continuous pressure over a large enough area. In some embodiments, the upper and lower circuits overlap partially in the up-and-down direction, i.e., in the packaging direction, but they will not overlap completely. It should be noted that the upper circuit and the lower circuit in the two conductive members are arranged in a staggered manner, which means that upper and lower circuits are entirely staggered, or only parts of them are staggered. For example, contact parts of the upper and lower circuits used for mutual connection are arranged in a staggered manner.

Specifically, referring to FIG. 2, the circuits in the two side regions are designed to be arranged in a staggered manner and connected in series in a bridge-type. In the two side regions of the pressure sensing pad, the upper circuit on the upper conductive member and the lower circuit on the lower conductive member are not directly aligned, but are arranged in a staggered manner. This design ensures that even when the user's palm or other small object briefly touches the surface of the pressure pad, the early warning signal will not be easily triggered, because the staggered arrangement of the upper and lower circuits forms the bridge-type series connection. The upper and lower circuits are not in simple face-to-face contact, but are in contact in a staggered manner through the hollow areas on the intermediate insulating foam, which means that the two circuits can only contact each other through these hollow gaps to form an effective electrical connection when they are subjected to continuous pressure over a large enough area.

Through the bridge-type series connection design, the care monitoring device can effectively filter out common slight pressure changes in daily activities, avoid unnecessary alarms, and reduce wear on the system caused by frequent false triggering, thereby extending the service life of the product.

In an embodiment, in the middle region, an upper circuit of the upper conductive member and a lower circuit of the lower conductive member are arranged in an aligned manner to form a contact-type series connection.

The contact-type series connection means that in the middle region of the pressure sensing pad, the upper circuit on the upper conductive member and the lower circuit on the lower conductive member are arranged in an aligned manner, which means that the upper circuit and the lower circuit are separated when there is no external force, but when appropriate pressure is applied, the two circuits can directly contact and form an electrical connection. This type of connection is called contact-type series connection.

Specifically, referring to FIG. 2, the circuits in the middle region are designed to be arranged in an aligned manner and connected in series in a contact-type. In the middle region of the pressure sensing pad, the upper circuit on the upper conductive member and the lower circuit on the lower conductive member are arranged in an aligned manner. In some embodiments, the upper and lower circuits are arranged in the same position and/or in the same shape, ensuring that they can accurately contact each other when the appropriate pressure is applied.

In an example, referring to FIG. 1, in the absence of external pressure, the upper circuit and the lower circuit are separated by a layer of insulating foam. In this case, the circuits are in an open state and no current exists. However, when the user's body (such as shoulders or hips) applies enough pressure to the middle region, the upper and lower circuits can directly contact each other through the hollow areas on the intermediate insulating foam, forming a complete microcircuit loop. This type of circuit connection achieved through physical contact is called contact-type series connection. Once the user leaves the middle region, the originally applied pressure disappears, and the upper and lower circuits will be separated under the natural tension of the foam, thus cutting off the electrical connection. The controller 106 detects this change in electrical connection and generates the target early warning signal accordingly, indicating that the user may have stood up, fallen, or left the safe area.

Through the contact-type series connection design, the care monitoring device can respond sensitively to changes in pressure in the middle region. As long as the user leaves the middle region, causing the originally applied pressure to disappear, the alarm will be triggered immediately. Once a real leaving event is determined, the care monitoring device can quickly send out the target early warning signal to notify the caregivers or family members to take timely action, thereby ensuring the safety of the user.

In an embodiment, the controller is configured to obtain a target duration in which the upper conductive assembly and the lower conductive assembly are electrically disconnected, and when the target duration is greater than a preset duration threshold, convert an electrical signal generated by the disconnection between the upper conductive assembly and the lower conductive assembly within the target duration into the target early warning signal.

The target duration refers to a time period the time period during which the controller detects that the upper conductive assembly and the lower conductive assembly remains disconnected after the disconnection occurs. In other words, the target duration is a period of time recorded by the controller during which the electrical connection between the upper conductive assembly and the lower conductive assembly is disconnected.

The preset duration threshold refers to a time limit preset by the care monitoring device, which is configured to determine whether a user leaving event actually occurs. It is a fixed reference time. For example, the preset duration threshold is set to 3 to 5 seconds (the specific value can be adjusted according to an actual need).

Specifically, in the middle region of the pressure sensing pad, when the user leaves the middle region, the originally applied pressure disappears, resulting in disconnection of the electrical connection between the upper conductive assembly and the lower conductive assembly. In this case, the controller will monitor and record the duration of the electrical disconnection, which is the target duration. In order to distinguish brief actions (such as turning over, arm supporting, etc.) from the real leaving event, the care monitoring device will set the preset duration threshold, for example, the preset duration threshold is set to 3 to 5 seconds (the specific value can be adjusted according to an actual need). The preset duration threshold is configured to determine whether the user leaving event actually occurs.

The controller is configured to compare the recorded target duration with the preset duration threshold. If the target duration is less than or equal to the preset duration threshold, the controller determines that the user had made a slight movement and does not trigger the alarm. If the target duration is greater than the preset duration threshold, the controller determines that the real leaving event has occurred and converts the electrical signal during this period into the target early warning signal. In some embodiments, once it is determined that the target duration is greater than the preset duration threshold, the controller will take the following actions, including converting the electrical signal generated by the disconnection between the upper conductive assembly and the lower conductive assembly within the target duration into the target early warning signal, and generating and sending the target early warning signal to the receiving host alarm, thus notifying the caregivers or family members to take necessary actions immediately, check the user's status and provide assistance.

By introducing the preset duration threshold, the care monitoring device can distinguish the brief actions (such as turning over, arm support, etc.) from the real leaving event. The alarm is triggered only when the electrical connection is disconnected for a duration longer than the preset threshold, which effectively reduces false alarms caused by daily activities and improves the reliability and user experience of care monitoring device. This mechanism ensures that the care monitoring device only alarms when the user actually leaves the middle region, avoiding unnecessary interference and allowing the caregivers or family members to have more trust in the prompt of the care monitoring device.

The present disclosure further provides a care monitoring system, including a care monitoring device according to any one of the above-described embodiments and an alarm device. The care monitoring device includes an upper conductive assembly, a lower conductive assembly, and an insulating assembly is arranged between the upper conductive assembly and the lower conductive assembly. The insulating assembly is provided with hollow areas. An end of the upper conductive assembly and/or an end of the lower conductive assembly is connected to a controller. When a human body applies pressure to the upper conductive assembly, the upper conductive assembly and the lower conductive assembly are electrically connected through the hollow areas in the insulating assembly, and the controller is configured to convert an electrical signal generated by the electrical connection into a pressure early warning signal.

The care monitoring device is configured to send the pressure early warning signal to the alarm device.

The alarm device is configured to receive the pressure early warning signal and issue an alarm prompt according to the pressure early warning signal.

The alarm device, as a part of the care monitoring system, is responsible for receiving the pressure early warning signal from the care monitoring device and issuing a corresponding alarm prompt according to the signal. The main function of the alarm device is to alert the caregivers or family members in a variety of ways when potential risks are detected, so that they can respond quickly.

In some embodiments, the alarm device includes a plurality of receiving host alarms, each of which can match a plurality of controllers of the pressure sensing pads.

In some embodiments, the alarm device is provided with a plurality of region indicator lights. The user can define the region represented by each indicator light according to needs, making it easier to identify which region has triggered the alarm.

In some embodiments, the receiving host alarm is small in design and has the functions of back clip, and lanyard, so it can be carried around, placed on a table or hung on a wall, ensuring that the caregivers can receive alarm information in a timely manner no matter where they are.

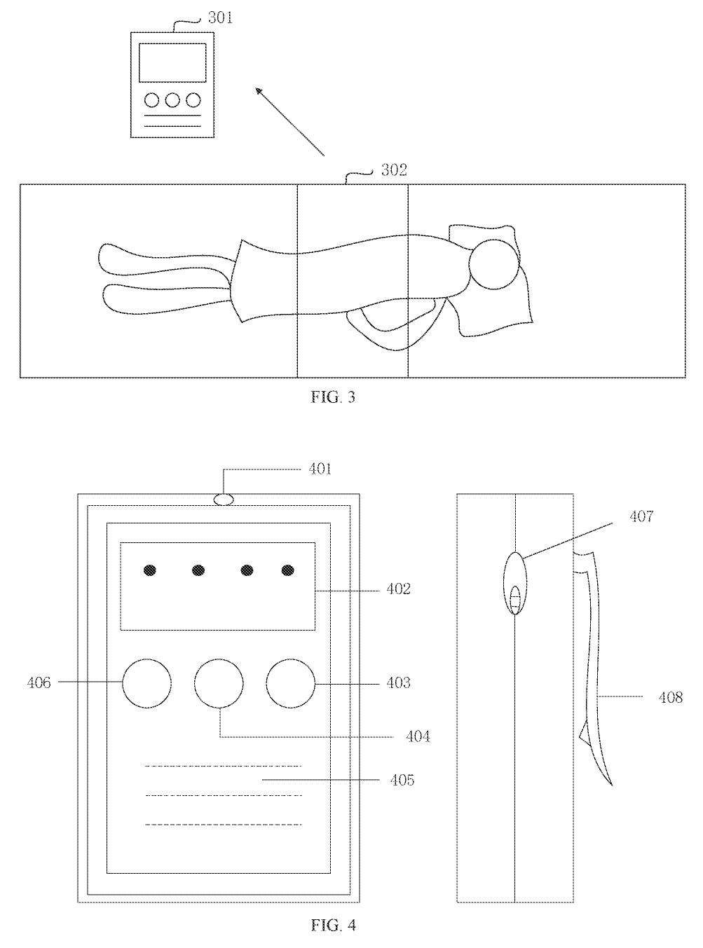

Specifically, referring to FIG. 3, the care monitoring system in the present disclosure is aimed to provide safety protection for people who are unable to move or who need care (such as elderly people, severely bedridden patients, dementia patients and young children). The care monitoring system consists of two major parts, i.e., an alarm device 301 and a care monitoring device 302.

The care monitoring device includes an upper conductive assembly, a lower conductive assembly, an insulating assembly and a controller. The upper conductive assembly, the lower conductive assembly, and the insulating assembly jointly form a pressure sensing pad. The upper conductive assembly and the lower conductive assembly are respectively located on the upper and lower sides of the pressure sensing pad, and are made of a specially designed conductive carbon circuit or a conductive aluminum foil. The insulating assembly is arranged between the upper conductive assembly and the lower conductive assembly, which is typically a layer of insulating foam with regularly arranged hollow areas thereon, allowing the upper conductive assembly and the lower conductive assembly to contact each other to form an electrical connection when pressure is applied. The controller is connected to an end of the upper conductive assembly or the lower conductive assembly and is responsible for monitoring the electrical connection status and converting the generated electrical signal into the pressure early warning signal.

In an example, when the human body applies pressure to the upper conductive assembly, the upper conductive assembly and the lower conductive assembly make physical contact through the hollow areas in the insulating assembly to form the electrical connection. After detecting this electrical connection, the controller converts the generated electrical signal into the pressure early warning signal.

The alarm device includes region indicator lights, back clips and receiving host alarms. The alarm device can receive the pressure early warning signal from the care monitoring device and issue a corresponding alarm prompt based on the signal. The receiving host alarm is provided with a plurality of region indicator lights. The user can define the region represented by each indicator light according to needs, making it easier to identify which region has triggered the alarm. The receiving host alarm is small in design and has the functions of back clip, and lanyard, so it can be carried around, placed on the table or hung on the wall, ensuring that the caregivers can receive alarm information in a timely manner no matter where they are.

In an example, when the user's body (such as shoulders or hips) applies pressure to the middle region of the pressure sensing pad, the upper conductive assembly and the lower conductive assembly contact through the hollow areas in the insulating assembly to form the electrical connection. After detecting the electrical connection, the controller converts the generated electrical signal into the pressure early warning signal. The care monitoring device sends the pressure early warning signal to the alarm device. The alarm device receives the pressure early warning signal and activates the corresponding prompt mechanism (such as sound, flashing light or vibration) according to the settings to notify the caregivers or family members. The caregivers or family members take necessary actions according to the alarm prompt, check the user's status and provide assistance. By manually pressing the “Stop Alarm” button, the alarm device can be reset to return the system to a standby mode, allowing monitoring to continue.

Through close cooperation between the care monitoring device and the alarm device, the care monitoring system of the present disclosure achieves accurate monitoring and timely warning of potential risks, not only improving the overall performance and reliability of the system, but also providing users with a safe, stable and convenient monitoring solution. Such a design can provide timely assistance at critical moments and effectively reduce the risk of accidents, and is especially suitable for those who require long-term monitoring.

In an embodiment, when the pressure applied by the human body to the upper conductive assembly of the care monitoring device disappears, the upper conductive assembly and the lower conductive assembly are electrically disconnected, and the controller is configured to generate a target early warning signal after the electrical connection is disconnected. The care monitoring device is further configured to send the target pressure early warning signal to the alarm device. The alarm device is further configured to receive the target early warning signal and issue an alarm prompt according to the target early warning signal.

Specifically, when the human body moves away from the monitoring area of the pressure pad, causing the pressure applied to the upper conductive assembly to disappear, the electrical connection between the upper conductive material (such as the conductive carbon circuit on the polyethylene-based film or the conductive aluminum foil) and the lower conductive assembly is disconnected due to the natural tension of the intermediate insulating foam. Once the disconnection of this electrical connection is detected, the built-in controller of the pressure sensing pad generates the target early warning signal, indicating that the user may have left the safety monitoring area, such as accidentally falling from a bed, chair or wheelchair.

The target early warning signal is transmitted to the receiving host alarm via wireless or wired means. After receiving the target early warning signal, the receiving host alarm will identify the signal type based on the preset specific coding and activate the corresponding alarm mechanism. The receiving host alarm has three prompt modes, i.e., sound, flashing light and vibration, ensuring that the caregivers or family members can be promptly notified and respond regardless of their situation (such as being asleep or having sensory impairment).

Through the above steps, the care monitoring device monitors the user's status changes and sending the early warning signal, while the alarm device receives this signal and performs the corresponding alarm action, thus forming a complete monitoring and alarm system aimed at ensuring the safety of the user and assisting the caregivers in effective monitoring.

In an embodiment, the alarm device includes a plurality of region indicator lights, and each of the region indicator lights corresponds to one care monitoring device. The alarm device is further configured to convert the target early warning signal received from the care monitoring device into a prompt signal according to a preset alarm prompt mode, and the prompt signal is configured to issue an alarm prompt according to the preset alarm prompt mode.

The region indicator light refers to a visual prompt component installed on the receiving host alarm. Each indicator light corresponds to a specific receiving host alarm, and one care monitoring device may include a plurality of receiving host alarms. These indicator lights are configured to visually display which monitoring device has sent a warning or alarm signal. The users can define the specific location or personnel represented by each indicator light according to their needs. For example, one indicator light may correspond to the bed in the bedroom, while another may correspond to the chair in the living room, and so on.

The alarm prompt mode refers to the mode used by the receiving host alarm to notify the caregivers or family members. In the present disclosure, the care monitoring device is designed with three different prompting modes: sound (auditory), flashing lights (visual), and vibration (tactile), which ensures that the users can receive the alarm information regardless of whether they have hearing or vision impairments or do not want to be disturbed when resting at night. The caregivers or family members can choose the alarm prompt mode that best suits their environment and personal preferences.

The prompt signal refers to an actual output generated by the receiving host alarm based on the target early warning signal received from the care monitoring device. This prompt signal will be converted according to the preset alarm prompt mode and sent to the caregivers or family members through the corresponding prompt mode (such as sound, flashing light or vibration). The prompt signal is designed to attract attention and prompt relevant personnel to take action to check or assist the monitored person.

Specifically, the alarm device is designed with the plurality of region indicator lights, and each indicator light is paired with one receiving host alarm, indicating that each pressure pad on a bed, chair or wheelchair can be associated with a unique indicator light on the receiving host alarm through its unique coding. The user can customize the specific locations or personnel represented by these indicator lights to clearly identify which region has triggered a warning or alarm event. Optionally, the alarm device includes a working/low power LED indicator light 401, a region indicator light 402, a stop button 403, a pairing button 404, a buzzer 405, a volume button 406, a power switch 407 and a back clip 408.

When receiving the target early warning signal transmitted from the care monitoring device, the receiving host alarm will convert the target early warning signal into an appropriate prompt signal according to the preset alarm prompt mode. The alarm prompt mode includes but is not limited to sound (auditory), flashing lights (visual) and vibration (tactile), so as to ensure that the caregivers or family members are notified in time regardless of the situation. The converted prompt signal is used for alarm prompting according to the preset alarm prompting mode.

In an example, if the target early warning signal indicates that the user may have left the safe area, the receiving host alarm may emit a rapid sound alarm and the corresponding region indicator light will start flashing. If the signal is an early warning signal, the receiving host alarm may emit a gentle sound or only the indicator light will light up. In this way, the caregivers or family members can respond quickly according to different prompt signals, check and assist the monitored person, thereby effectively preventing potential safety risks.

By pairing the plurality of region indicator lights with care monitoring devices in different locations on a one-to-one basis, the caregivers or family members can quickly determine which specific device has issued a warning or alarm signal, facilitating quickly locating the location of the incident, such as which bed or chair occupant has an abnormal situation, thereby improving response efficiency. Moreover, according to different user needs and environmental conditions, the most appropriate alarm prompt mode (sound, flashing light, vibration) can be selected to ensure that the caregivers or family members can be effectively notified even in the case of hearing or visual impairments. In addition, at night or in a quiet environment, a silent mode with vibration or light prompts can be selected to avoid disturbing other people's rest.

Although the embodiments of the present disclosure have been shown and described, it should be understood that a person skilled in the art may make various changes, modifications, substitutions and variations to these embodiments without departing from the principles and spirit of the present disclosure, and the scope of the present disclosure is defined by the appended claims and their equivalents.

What is claimed is:

1. A care monitoring device, comprising an upper conductive assembly, a lower conductive assembly, an insulating assembly arranged between the upper conductive assembly and the lower conductive assembly, and a controller, the insulating assembly being provided with a hollow area,
wherein when a human body applies pressure to the upper conductive assembly, the upper conductive assembly and the lower conductive assembly are electrically connected through the hollow area in the insulating assembly, and the controller is configured to convert an electrical signal generated by the electrical connection into a pressure early warning signal.

2. The care monitoring device according to claim 1, wherein when a pressure applied by the human body to the upper conductive assembly disappears, the upper conductive assembly and the lower conductive assembly are electrically disconnected, and the controller is configured to generate a target early warning signal after the electrical connection is disconnected.

3. The care monitoring device according to claim 2, wherein the upper conductive assembly comprises an upper conductive member and an upper plastic member, and the upper plastic member is located above the upper conductive member; and
the lower conductive assembly comprises a lower conductive member and a lower plastic member, the lower plastic member is located below the lower conductive member, and the upper plastic member and the lower plastic member are configured to package the upper conductive member, the lower conductive member, and the insulating assembly.

4. The care monitoring device according to claim 2, wherein the upper conductive assembly comprises an upper conductive member, the lower conductive assembly comprises a lower conductive member, the care monitoring device is divided into two side regions and a middle region, the two side regions are configured to generate the pressure early warning signal, and the middle region is configured to generate the target early warning signal.

5. The care monitoring device according to claim 4, wherein in the two side regions, an upper circuit of the upper conductive member and a lower circuit of the lower conductive member are arranged in a staggered manner to form a bridge-type series connection.

6. The care monitoring device according to claim 5, wherein the upper circuit of the upper conductive member and the lower circuit of the lower conductive member overlap partially in a packaging direction of the packaged upper conductive member, the insulating assembly and the lower conductive member.

7. The care monitoring device according to claim 4, wherein in the middle region, an upper circuit of the upper conductive member and a lower circuit of the lower conductive member are arranged in an aligned manner to form a contact-type series connection.

8. The care monitoring device according to claim 4, wherein the side regions are configured to match positions of hands or arms of a user lying in bed or sitting on a chair, and the middle region is configured to match positions of shoulders or hips of the user, when the care monitoring device is used on a bed or a chair.

9. The care monitoring device according to claim 2, wherein the controller is configured to obtain a target duration in which the upper conductive assembly and the lower conductive assembly are electrically disconnect, and when the target duration is greater than a preset duration threshold, convert an electrical signal generated by the disconnection between the upper conductive assembly and the lower conductive assembly within the target duration into the target early warning signal.

10. The care monitoring device according to claim 1, wherein at least one of the upper conductive assembly and the lower conductive assembly is connected to the controller.

11. The care monitoring device according to claim 1, wherein the insulating assembly comprises an insulating foam, and the hollow area comprises gaps arranged in the insulating foam.

12. A care monitoring system, comprising a care monitoring device and an alarm device, the care monitoring device comprising an upper conductive assembly, a lower conductive assembly, an insulating assembly arranged between the upper conductive assembly and the lower conductive assembly, and a controller, the insulating assembly being provided with a hollow area,
wherein when a human body applies pressure to the upper conductive assembly, the upper conductive assembly and the lower conductive assembly are electrically connected through the hollow areas in the insulating assembly, and the controller is configured to convert an electrical signal generated by the electrical connection into a pressure early warning signal;
the care monitoring device is configured to send the pressure early warning signal to the alarm device; and
the alarm device is configured to receive the pressure early warning signal and issue an alarm prompt according to the pressure early warning signal.

13. The care monitoring system according to claim 12, wherein when the pressure applied by the human body to the upper conductive assembly of the care monitoring device disappears, the upper conductive assembly and the lower conductive assembly are electrically disconnected, and the controller is configured to generate a target early warning signal after the electrical connection is disconnected;
the care monitoring device is further configured to send the target pressure early warning signal to the alarm device; and
the alarm device is further configured to receive the target early warning signal and issue an alarm prompt according to the target early warning signal.

14. The care monitoring system according to claim 13, wherein the alarm device comprises a plurality of region indicator lights, and each of the region indicator lights corresponds to one care monitoring device; and
the alarm device is further configured to convert the target early warning signal received from the care monitoring device into a prompt signal according to a preset alarm prompt mode, and the prompt signal is configured to issue an alarm prompt according to the preset alarm prompt mode.

15. The care monitoring system according to claim 13, wherein the upper conductive assembly comprises an upper conductive member and an upper plastic member, and the upper plastic member is located above the upper conductive member; and
the lower conductive assembly comprises a lower conductive member and a lower plastic member, the lower plastic member is located below the lower conductive member, and the upper plastic member and the lower plastic member are configured to package the upper conductive member, the lower conductive member, and the insulating assembly.

16. The care monitoring system according to claim 13, wherein the upper conductive assembly comprises an upper conductive member, the lower conductive assembly comprises a lower conductive member, the care monitoring device is divided into two side regions and a middle region, the two side regions are configured to generate the pressure early warning signal, and the middle region is configured to generate the target early warning signal.

17. The care monitoring system according to claim 16, wherein in the two side regions, an upper circuit of the upper conductive member and a lower circuit of the lower conductive member are arranged in a staggered manner to form a bridge-type series connection.

18. The care monitoring system according to claim 17, wherein the upper circuit of the upper conductive member and the lower circuit of the lower conductive member overlap partially in a packaging direction of the packaged upper conductive member, the insulating assembly and the lower conductive member.

19. The care monitoring system according to claim 16, wherein in the middle region, an upper circuit of the upper conductive member and a lower circuit of the lower conductive member are arranged in an aligned manner to form a contact-type series connection.

20. The care monitoring system according to claim 13, wherein the controller is configured to obtain a target duration in which the upper conductive assembly and the lower conductive assembly are electrically disconnect, and when the target duration is greater than a preset duration threshold, convert an electrical signal generated by the disconnection between the upper conductive assembly and the lower conductive assembly within the target duration into the target early warning signal.
